(19) United States
(12) Patent Application Publication
(10) Pub. No.: US 2025/0058042 A1
(43) Pub. Date: Feb. 20, 2025

(54) DYNAMIC ADJUSTMENT OF A CORRECTION FACTOR OF A USER BASED ON GLUCOSE VALUES

(71) Applicant: INSULET CORPORATION, Acton, MA (US)

(72) Inventors: Joon Bok LEE, Acton, MA (US); Yibin ZHENG, Hartland, WI (US); William WHITELEY, Knoxville, TN (US); Jason O’CONNOR, Acton, MA (US)

(21) Appl. No.: 18/800,698

(22) Filed: Aug. 12, 2024

Related U.S. Application Data
(60) Provisional application No. 63/519,336, filed on Aug. 14, 2023.

(51) Int. Cl. A61M 5/142

(52) U.S. Cl. CPC A61M 5/142; A61M 2202/0007; A61M 2202/04; A61M 2205/10; A61M 2205/3303; A61M 2205/3327; A61M 2205/3553; A61M 2205/3561; A61M 2205/3592; A61M 2205/502; A61M 2205/52; A61M 2230/005; A61M 2230/201

(57) ABSTRACT

The exemplary embodiments may provide a correction factor (CF) for a user that is dynamically adjustable based on current glucose level. In some exemplary embodiments, the CF of the user is adjusted based on the current glucose level of the user. As the current glucose level of the user increases to be above a threshold, the CF may be adjusted to reflect a decrease in the insulin sensitivity of the user. This adjustment may cause the control system to increase the amount of insulin delivered to the user as the glucose level of the user exceeds the threshold. Similarly, the CF of the user may be adjusted based on the current target glucose level of the user or based on a combination of the current glucose level of the user and the current target glucose level of the user.

### CROSS-REFERENCE TO RELATED APPLICATIONS

This application claims priority to and the benefit of U.S. Provisional Application No. 63/519,336, filed Aug. 14, 2023, the entirety of which is incorporated herein by reference.

### BACKGROUND

Automated insulin delivery systems, like on-body insulin pumps, attempt to control a glucose level of a user by delivering insulin based on glucose level feedback. In determining how much insulin to deliver, a control system for such an insulin delivery system may use a correction factor (CF) of the user. The CF of the user specifies how much 1 unit of insulin will reduce a glucose level of the user. The CF is used to determine the extent of an insulin deficiency of the user or the extent of an excess relative to a glucose level target. Conventionally, the numerical value of the CF is constant across all conditions.

Unfortunately, the insulin sensitivity of the user varies. Hence, the use of a constant CF of the user may produce unsatisfactory results. For instance, insulin sensitivity tends to decrease as glucose levels of the user increase, for example, above a glucose level target. Hence, for such higher glucose levels, more insulin is required to reduce the glucose level than if the glucose level of the user is at lower levels.

This disadvantage associated with keeping the CF of the user constant is further complicated by biasing the CF value of the user to avoid hypoglycemia. Hence, the CF is typically set with a bias toward delivering less insulin to the user by setting the CF to reflect a higher insulin sensitivity than the CF would otherwise be set at.

### SUMMARY

In accordance with a first inventive aspect, a medicament delivery system for delivering medicament to a user includes a non-transitory storage medium storing computer programming instructions. The system may also include a processor configured for executing the computer programming instructions. Executing the computer programming instructions causes the processor to determine a current glucose level of the user. Based on the determined current glucose level of the user, the processor modifies a current correction factor of the user and determines a dose of medicament to deliver to the user using the modified correction factor.

The medicament delivery system may include a medicament delivery device, and the processor may be part of the medicament delivery device. The computer programming instructions, when executed by the processor, may cause the processor to define a range of glucose level values for which the current correction factor of the user will be modified and may cause the processor to determine whether the current glucose level of the user falls within the range. The modified correction factor may be determined based upon where the current glucose level of the user falls within the range. The modified correction factor may, for example, be set at a value equal to a numerator divided by total daily insulin (TDI) of the user. The numerator may be adjusted from a standard value to a modified value to modify the current correction factor of the user. The numerator, in some instances, may only be adjusted to a modified value that is in a range extending from the standard value to a minimum permitted value. The modified correction value may be calculated using a scaled value relative to the standard value based upon where the current glucose level value of the user falls within the range. The modified scaled value may be scaled linearly or quadratically relative to the standard value, in some embodiments.

In accordance with another inventive facet, a medicament delivery system for delivering medicament to a user includes a non-transitory storage medium storing computer programming instructions. The system may also include a processor configured for executing the computer programming instructions to cause the processor to determine a current target glucose level of the user and based on the current target glucose level of the user, to modify a current correction factor of the user. The computer programming instruction when executed may also cause the processor to determine a dose of medicament to deliver to the user using the modified correction factor.

The computer programming instructions, when executed by the processor, may cause the processor to define a range of target glucose levels for which the current correction factor of the user will be modified and to determine whether the current target glucose level of the user falls within the range. The modified correction factor may, for example, be set at a value equal to a numerator divided by the TDI of the user. The numerator may be adjusted from a standard value to a modified value to modify the current correction factor of the user. The numerator may, in some instances, only be adjusted to a modified value that is in a range extending from the standard value to a minimum permitted value. The modified correction value may be calculated using a scaled value relative to the standard value based upon where the current glucose level value of the user falls within the range. The modified scaled value may be scaled linearly or quadratically relative to the standard value, in some embodiments.

In accordance with another inventive facet, a medicament delivery system for delivering medicament to a user may include a non-transitory storage medium storing computer programming instructions and a processor configured for executing the computer programming instructions. Executing the computer programming instructions may cause the processor to determine a current glucose level of the user and to determine a current target glucose level of the user. Executing the computer programming instructions also may cause the processor, based on the current glucose level of the user and the current target glucose level of the user, to modify a current correction factor of the user and to determine a dose of medicament to deliver to the user using the modified correction factor.

The modified correction factor may be a scaled version of the current correction factor wherein the scaling is based on both where the current glucose level of the user falls within a range of glucose level values and where the current target glucose level value falls with a range of target glucose level values. The medicament may comprise insulin. The correction factor may, in some embodiments, only be modified if the current glucose level of the user exceeds a threshold value or, in some embodiments, a current glucose level target of the user.

### BRIEF DESCRIPTION OF THE DRAWINGS

FIG. 1 depicts a diagram of a medicament delivery system of exemplary embodiments.

FIG. 2A depicts a flowchart of illustrative steps that may be performed in exemplary embodiments to adjust and use CF of a user in a medicament delivery system.

FIG. 2B depicts a flowchart of illustrative steps that may be performed in exemplary embodiments as part of a CF adjustment loop.

FIG. 3 depicts a flowchart of illustrative steps that may be performed in exemplary embodiments to potentially adjust the CF of the user based on current glucose level of the user.

FIG. 4 depicts a flowchart of illustrative steps that may be performed in exemplary embodiments when adjusting CF of the user based on the current glucose level of the user.

FIG. 5 depicts a plot of numerator values and the scaling of numerator values within a range in exemplary embodiments.

FIG. 6 depicts a flowchart of illustrative steps that may be performed in exemplary embodiments to quadratically scale the numerator in modifying the current CF of the user.

FIG. 7 depicts a flowchart of illustrative steps that may be performed to calculate G′(i) in some exemplary embodiments.

FIG. 8A depicts a flowchart of illustrative steps that may be performed in exemplary embodiments in prohibiting adjustment of the CF of the user following a meal.

FIG. 8B depicts a flowchart of illustrative steps that may be performed in exemplary embodiments to adjust correction boluses after meal ingestion.

FIG. 9 depicts a flowchart of illustrative steps that may be performed in exemplary embodiments to adjust CF of the user based on current target glucose level.

FIG. 10 depicts a flowchart of illustrative steps that may be performed in exemplary embodiments in adjusting the CF of the user based on current target glucose level.

FIG. 11 depicts a flowchart of illustrative steps that may be performed in exemplary embodiments in adjusting the CF of the user based on current glucose level and current target glucose level.

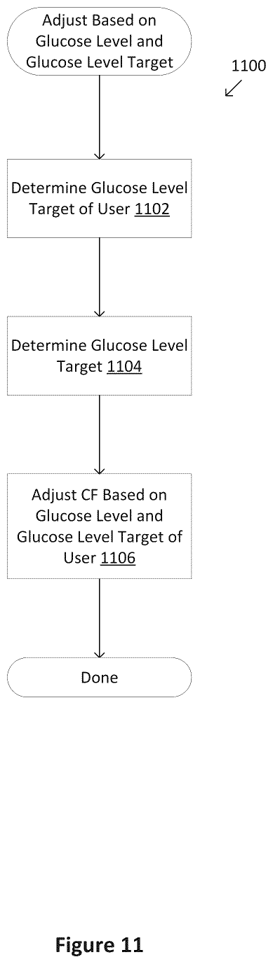

FIG. 12 depicts a flowchart of illustrative steps that may be performed in exemplary embodiments per a first option to adjust the CF of the user based on both current glucose level of the user and current target glucose level.

FIG. 13 depicts a flowchart of illustrative steps that may be performed in exemplary embodiments per a second option to adjust the CF of the user based on both current glucose level of the user and current target glucose level.

FIG. 14 depicts a flowchart of illustrative steps that may be performed in exemplary embodiments per a third option to adjust the CF of the user based on both current glucose level of the user and current target glucose level.

FIG. 15 depicts a flowchart of illustrative steps that may be performed in exemplary embodiments to calculate th_{mid}.

### DETAILED DESCRIPTION

The exemplary embodiments may provide a correction factor (CF) for a user that is dynamically adjustable based on a current glucose level, a trend of glucose levels (e.g., a mean or median of recent glucose levels), and/or a current target glucose level or. In some exemplary embodiments, the CF of the user is adjusted based on the current glucose level of the user. As the current glucose level value of the user increases to be above a threshold, the CF may be adjusted to reflect a decrease in the insulin sensitivity of the user. This adjustment may cause the control system to increase the amount of insulin delivered to the user as the glucose level of the user exceeds the threshold.

In some exemplary embodiments, the adjustment to the CF of the user may be according to a scale based upon where the current glucose level of the user lies in a range between a lower glucose level threshold and an upper glucose level threshold. In some exemplary embodiments, the adjustment of the CF of the user may be linearly scaled based upon where a current glucose level of the user lies in the range. In other exemplary embodiments, the CF of the user may be quadratically scaled. Other scaling approaches may be used or no scaling may be used in other exemplary embodiments.

In some exemplary embodiments, the CF of the user is dynamically adjusted based on the current glucose level target rather than the current glucose level of the user. The CF may be scaled based upon where the current glucose level target lies in a specified range. The scaling may be linear or quadratic, for example.

In these exemplary embodiments, the dynamic adjustment of the CF of the user helps to provide better glucose level control for elevated glucose levels of the user. The elevated glucose level of the user may be more quickly brought back into a desirable range than with a conventional fixed CF approach.

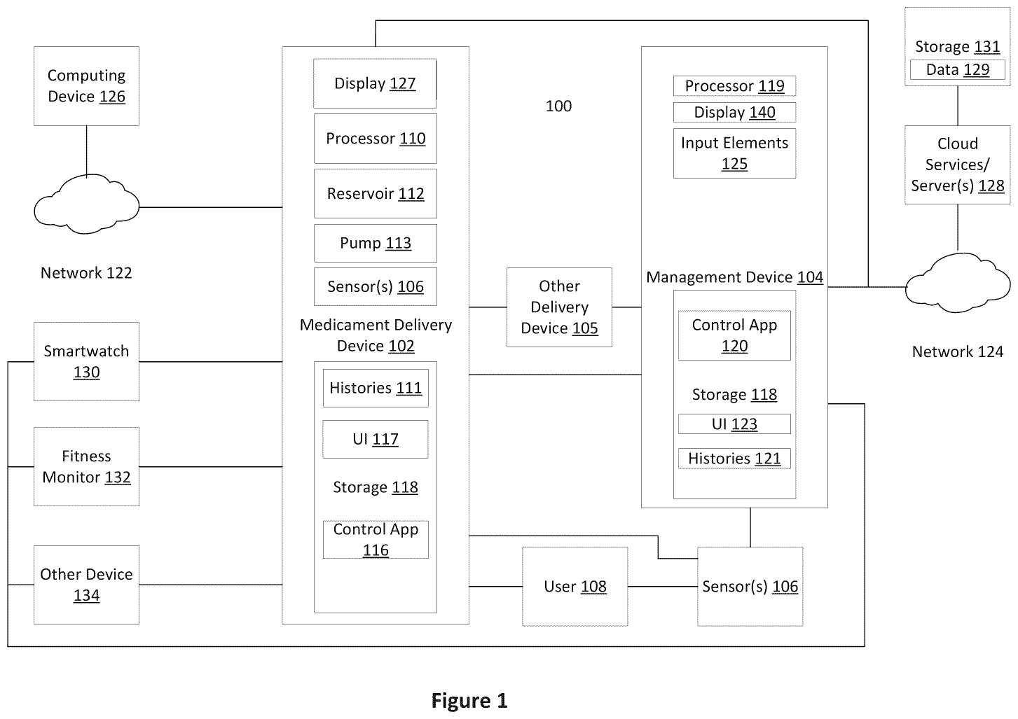

FIG. 1 depicts a block diagram of an illustrative medicament delivery system 100 that is suitable for delivering a medicament, such as insulin, to a user 108 in accordance with the exemplary embodiments. The medicament delivery system 100 may include a medicament delivery device 102. The medicament delivery device 102 may be a wearable device that is worn on the body of the user 108 or carried by the user. The medicament delivery device 102 may be directly coupled to the user 108 (e.g., directly attached to a body part and/or skin of the user 108 via an adhesive or the like) with no tubes and an infusion location directly under the medicament delivery device 102, or carried by the user 108 (e.g., on a belt or in a pocket) with the medicament delivery device 102 connected to an infusion site where the medicament is injected using a needle and/or cannula. A surface of the medicament delivery device 102 may include an adhesive to facilitate attachment to the user 108.

The medicament delivery device 102 may include a processor 110. The processor 110 may be, for example, a microprocessor, a logic circuit, a field programmable gate array (FPGA), an application specific integrated circuit (ASIC) or a microcontroller. The processor 110 may maintain a date and time as well as other functions (e.g., calculations or the like). The processor 110 may be operable to execute a control application 116 encoded in computer programming instructions stored in the storage 114 that enables the processor 110 to direct operation of the medicament delivery device 102. The control application 116 may be a single program, multiple programs, modules, libraries or the like. The processor 110 also may execute computer programming instructions stored in the storage 114 for a user interface (UI) 117 that may include one or more display screens shown on display 127. The display 127 may display information to the user 108 and, in some instances, may receive input from the user 108, such as when the display 127 is a touchscreen.

The control application 116 may control delivery of the medicament to the user 108 per a control approach like that described herein. The control application may use a glucose prediction model as described below for predicting future glucose levels of the user 108. The storage 114 may hold histories 111 for a user, such as a history of basal deliveries, a history of bolus deliveries, and/or other histories, such as a meal event history, exercise event history, glucose level history, other analyte level history, and/or the like. In addition, the processor 110 may be operable to receive data or information. The storage 114 may include both primary memory and secondary memory. The storage 114 may include random access memory (RAM), read only memory (ROM), optical storage, magnetic storage, removable storage media, solid state storage or the like.

The medicament delivery device 102 may include a tray or cradle and/or one or more housings for housing its various components including a pump 113, a power source (not shown), and a reservoir 112 for storing medicament for delivery to the user 108. A fluid path to the user 108 may be provided, and the medicament delivery device 102 may expel the medicament from the reservoir 112 to deliver the medicament to the user 108 using the pump 113 via the fluid path. The fluid path may, for example, include tubing coupling the medicament delivery device 102 to the user 108 (e.g., tubing coupling a cannula to the reservoir 112), and may include a conduit to a separate infusion site. The medicament delivery device 102 may have operational cycles, such as every 5 minutes, in which basal doses of medicament are calculated and delivered as needed. These steps are repeated for each cycle.

There may be one or more communications links with one or more devices physically separated from the medicament delivery device 102 including, for example, a management device 104 of the user 108 and/or a caregiver of the user 108, sensor(s) 106, a smartwatch 130, a fitness monitor 132 and/or another variety of device 134. The communication links may include any wired or wireless communication links operating according to any known communications protocol or standard, such as Bluetooth®, Wi-Fi, a near-field communication standard, a cellular standard, or any other wireless protocol.

The medicament delivery device 102 may interface with a network 122 via a wired or wireless communications link. The network 122 may include a local area network (LAN), a wide area network (WAN), a cellular network, a Wi-Fi network, a near field communication network, or a combination thereof. A computing device 126 may be interfaced with the network 122, and the computing device may communicate with the medicament delivery device 102.

The medicament delivery system 100 may include one or more sensor(s) 106 for sensing the levels of one or more analytes. The sensor(s) 106 may be coupled to the user 108 by, for example, adhesive or the like and may provide information or data on one or more medical conditions, physical attributes, or analyte levels of the user 108. The sensor(s) 106 may be physically separate from the medicament delivery device 102 or may be an integrated component thereof. The sensor(s) 106 may include, for example, glucose monitors, such as continuous glucose monitors (CGM's) and/or non-invasive glucose monitors. The sensor(s) 106 may include ketone sensors, other analyte sensors, heart rate monitors, breathing rate monitors, motion sensors, temperature sensors, perspiration sensors, blood pressure sensors, alcohol sensors, or the like. Some sensors 106 may also detect characteristics of components of the medicament delivery device 102. For instance, the sensors 106 in the medicament delivery device may include voltage sensors, current sensors, temperature sensors and the like.

The medicament delivery system 100 may or may not also include a management device 104. In some embodiments, no management device is needed as the medicament delivery device 102 may manage itself. The management device 104 may be a special purpose device, such as a dedicated personal diabetes manager (PDM) device. The management device 104 may be a programmed general-purpose device, such as any portable electronic device including, for example, a dedicated controller, such as a processor, a micro-controller, or the like. The management device 104 may be used to program or adjust operation of the medicament delivery device 102 and/or the sensor(s) 106. The management device 104 may be any portable electronic device including, for example, a dedicated device, a smartphone, a smartwatch, or a tablet. In the depicted example, the management device 104 may include a processor 119 and a storage 118. The processor 119 may execute processes to manage a user's glucose levels and to control the delivery of the medicament to the user 108. The medicament delivery device 102 may provide data from the sensors 106 and other data to the management device 104. The data may be stored in the storage 118. The processor 119 may also be operable to execute programming code stored in the storage 118. For example, the storage 118 may be operable to store one or more control applications 120 for execution by the processor 119. Storage 118 may also be operable to store historical information such as medicament delivery information, analyte level information, user input information, output information, or other historical information. The control application 120 may be responsible for controlling the medicament delivery device 102, such as by controlling the automated medicament delivery (AMD) (or, for example, automated insulin delivery (AID)) of medicament to the user 108. The storage 118 may store the control application 120, histories 121 like those described above for the medicament delivery device 102, and other data and/or programs.

A display 140, such as a touchscreen, may be provided for displaying information. The display 140 may display user interface (UI) 123. The display 140 also may be used to receive input, such as when the display is a touchscreen. The management device 104 may further include input elements 125, such as a keyboard, button, knobs, or the like, for receiving input of the user 108.

The management device 104 may interface with a network 124, such as a LAN or WAN or combination of such networks, via wired or wireless communication links. The management device 104 may communicate over network 124 with one or more servers or cloud services 128. Data, such as sensor values, may be sent, in some embodiments, for storage and processing from the medicament delivery device 102 directly to the cloud services/server(s) 128 or instead from the management device 104 to the cloud services/server(s) 128.

Other devices, like smartwatch 130, fitness monitor 132 and device 134 may be part of the medicament delivery system 100. These devices 130, 132 and 134 may communicate with the medicament delivery device 102 and/or management device 104 to receive information and/or issue commands to the medicament delivery device 102. These devices 130, 132 and 134 may execute computer programming instructions to perform some of the control functions otherwise performed by processor 110 or processor 119, such as via control applications 116 and 120. These devices 130, 132 and 134 may include displays for displaying information. The displays may show a user interface for providing input by the user 108, such as to request a change or pause in dosage, or to request, initiate, or confirm delivery of a bolus of medicament, or for displaying output, such as a change in dosage (e.g., of a basal delivery amount) as determined by processor 110 or management device 104. These devices 130, 132 and 134 may also have wireless communication connections with the sensor 106 to directly receive analyte measurement data. Another delivery device 105, such as a medicament delivery pen (such as an insulin pen), may be accounted for (e.g., in determining insulin on board (IOB)) or may be provided for also delivering medicament to the user 108.

The functionality described herein for the exemplary embodiments may be under the control of or performed by the control application 116 of the medicament delivery device 102 or the control application 120 of the management device 104. In some embodiments, the functionality wholly or partially may be under the control of or performed by the cloud services/servers 128, the computing device 126 or by the other enumerated devices, including smartwatch 130, fitness monitor 132 or another wearable device 134. The instructions may also be performed by a plurality of processors for example in a distributed computer system. The computer programs of the present disclosure may be for example preinstalled on, or downloaded to the medicament delivery device, management device, fluid delivery device, e.g. their storage.

In the closed loop mode, the control application 116, 120 determines the medicament delivery amount for the user 108 on an ongoing basis based on a feedback loop. For a medicament delivery device that uses insulin, for example, the aim of the closed loop mode is to have the user's glucose level at a target glucose level or within a target glucose range.

In some embodiments, the medicament delivery device 102 need not deliver one medicament alone. Instead, the medicament delivery device 102 may one medicament, such as insulin, for lowering glucose levels of the user 108 and also deliver another medicament, such as glucagon, for raising glucose levels of the user 108. The medicament delivery device 102 may deliver a glucagon-like peptide (GLP)-1 receptor agonist medicament for lowering glucose or slowing gastric emptying, thereby delaying spikes in glucose after a meal. The medicament delivery device 102 may deliver a gastric inhibitory polypeptide (GIP) or a dual GIP-GLP receptor agonist. In other embodiments, the medicament delivery device 102 may deliver pramlintide, or other medicaments that may substitute for insulin. In other embodiments, the medicament delivery device 102 may deliver concentrated insulin. In some embodiments, the medicament or medicament delivered by the medicament delivery device may be a coformulation of two or more of those medicaments identified above. In a preferred embodiment, the medicament delivery device delivers insulin; accordingly, reference will be made throughout this application to insulin and an insulin delivery device, but one of ordinary skill in the art would understand that medicaments other than insulin can be delivered in lieu of or in addition to insulin.

Insulin deliveries to the user 108 may be bolus insulin deliveries or basal insulin deliveries. Bolus insulin deliveries tend to be to offset the expected rise in glucose level of the user 108 from ingesting a meal or for correcting a persistently elevated glucose level (i.e., one that is persistently higher than a target glucose level). Boluses tend to be one time deliveries for offsetting a meal or for correcting a glucose level and tend to be larger than bolus insulin deliveries. Insulin boluses may be delivered manually by the user 108, such as via a syringe, or may, in some exemplary embodiments, be delivered by the medicament delivery device 102.

Basal insulin doses tend to be smaller than insulin bolus doses and are delivered periodically, such as once each operational cycle of the control approach of the medicament delivery device 102 (e.g., every 5 minutes). In some embodiments, each cycle has a length between about 30 seconds to about 30 minutes, more specifically between about 1.5 minutes to about 10 minutes and in particular between about 3 minutes to about 9 minutes. The aim of the basal insulin deliveries is to keep the user's glucose level within a target range that is desirable using small ongoing insulin doses.

The control approach of the exemplary embodiments that is performed by the control application 116 or 120, which may select a suitable insulin dose among candidate basal insulin delivery doses based on a cost function. A typical conventional cost function is:

$$J ( k ) = Q {\sum }_{i = k + 1}^{P} {( G ( i ) - SP ( i ) )}^{2} + R {\sum }_{i = k + 1}^{C} {( I ( i ) - b ( i ) )}^{2} \qquad ( Equation 1 )$$

where J(k) is the cost of a specified insulin dose for cycle k, Q Σᵢ₌ₖ₊₁^{P}(G(i)−SP(i))² is the glucose cost component, and R Σᵢ₌ₖ₊₁^{C}(I(i)−b(i))² is the insulin cost component. Q is a weight coefficient for the glucose cost component. The glucose cost component represents the weighted sum of the deviations squared in the glucose level of the user 108 (G(i)) over a future time horizon (cycles k+1 to P) relative to a target glucose level SP(i) if the specified basal insulin dose is delivered, R is a weight coefficient for the insulin cost component. The insulin cost component represents the costs of the squares of the deviations in insulin delivery amounts (I(i)) delivered over a time period (cycles k+1 to C) in the future relative to an ideal basal insulin dose (b(i)).

A correction factor (CF) for a user refers to how much 1 unit of insulin will reduce a user's glucose level. The CF for a user plays a role in determining a dosage size of a correction bolus, and may be part of a full bolus calculation that includes both correction and meal bolus components. The CF for a user may also be utilized by automated insulin delivery algorithms when calculating safety constraints and determining optimal insulin delivery requirements. One equation for calculating the size of the correction bolus is:

$${B}_{c} ( i ) = \frac{G ( i ) - SP ( i )}{CF} \qquad ( Equation 2 )$$

where B_{c}(i) is the size of the correction bolus at cycle i, G(i) is the glucose level of the user at cycle i, SP(i) is the setpoint, aka glucose level target, of the user, and CF is the correction factor of the user. The correction factor for a user is typically calculated by applying the 1800 rule as follows:

$$CF = \frac{1800}{TDI} \qquad ( Equation 3 )$$

where TDI is the total daily insulin of the user (i.e., daily bolus total+daily basal total). The CF may also be used in calculating the insulin on board (IOB) of the user that is required to bring the glucose level of the user to target SP. IOB refers to the amount of insulin that is still working in the body of the user but that has not yet affected the glucose level of the user. The required IOB at cycle i, designated as IOB_{req}(i), may be calculated as:

$${IOB}_{req} ( i ) = \frac{G ( i ) - SP ( i )}{CF} - IOB ( i ) \qquad ( Equation 4 )$$

where IOB(i) is the IOB of the user at cycle i.

As was mentioned above, conventionally the CF of the user is held constant. The exemplary embodiments recognize that the CF varies significantly with higher glucose levels of the user. For example, the insulin sensitivity may increase with increasing glucose levels. Therefore, more units of insulin may be required to reduce the blood glucose level by 1 mg/dL at higher blood glucose levels compared to lower blood glucose levels. In particular, insulin sensitivity may increase above euglycemia, i.e., at hypoglycemia. Additionally, constant CF are typically biased to prevent hypoglycemia, i.e. they are set to represent a higher insulin sensitivity than would be ideal for the user (or for example determined by the 1800-rule). The typically set constant CF therefore tend to lean towards delivering less insulin to the user than calculated as ideal. However, this bias of constant CFs representing higher insulin sensitivities further increases the problem of lower insulin sensitivities at higher glucose levels. Hence, the exemplary embodiments may allow the CF to vary as glucose levels get to elevated levels. The exemplary embodiments may provide improved control of higher glucose levels, while not (significantly) increasing the risk of hypoglycemia.

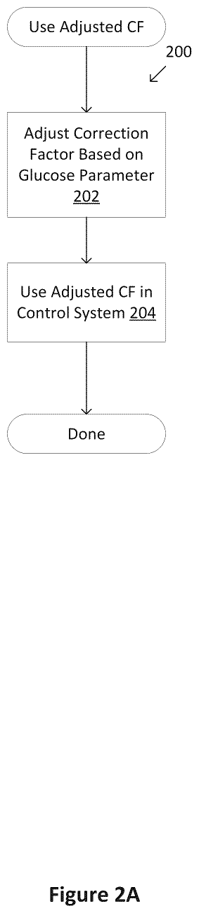

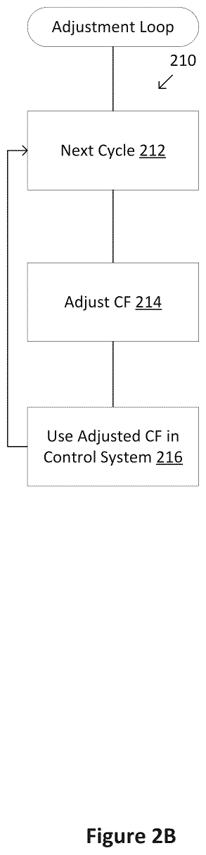

FIG. 2A depicts a flowchart 200 of illustrative steps that may be performed in exemplary embodiments in using such adjusted CF values in the control application 116 or 120. Initially, at 202, the CF of the user may be adjusted based on a glucose parameter, as detailed below. The glucose parameter, may be, for instance, a current glucose level of the user or a current glucose level target of the user. In some embodiments, the current glucose level of the user may be the last glucose level received, e.g. from the sensor 106. In some embodiments, the current glucose level of the user may be a weighted average (i.e., a weighted mean) or a median of the two to five most recent glucose levels received, in particular the two to three most recent glucose levels received. The weighting may prioritize more recent. In some embodiments, the current glucose level of the user may be a weighted average of the glucose levels received in a time interval preceding the current time, wherein the time interval preceding the current time has a length of 2 to 15 minutes, in particular 4 to 10 minutes. The weighting may prioritize more recently received glucose levels. Preferably, the current glucose level the is most recently received glucose level of the user. Correspondingly, the current glucose level target of the user may be the glucose level target currently used by the control application 116 or 120. In particular, the term “current target glucose level” as used herein, may refer to the glucose level currently used as target value by the cost function, i.e., wherein the glucose cost of the cost function is 0 when the candidate dose would result in the glucose level of the user being the target value. At 204, the adjusted CF may then be used by the control application or other mechanism in the control system to determine the required IOB for a basal insulin delivery dose determination or correction bolus determination, such as discussed above relative to Equations 2 and 4.

The adjustment of the CF of the user may occur regularly at periodic intervals or may be triggered by an event, such as the glucose level of the user being above a threshold. FIG. 2B depicts a flowchart of illustrative steps that may be performed in exemplary embodiments to adjust the CF each cycle. At 212, a next cycle is reached. The cycle may be an operational cycle of the medicament delivery device 102. Each cycle may represent a five-minute period of time, for example, or other periods of different lengths. At 214, the CF of the user may be adjusted as described below. At 216, the adjusted CF is used by the control system 216. The process may be repeated when the next cycle is reached.

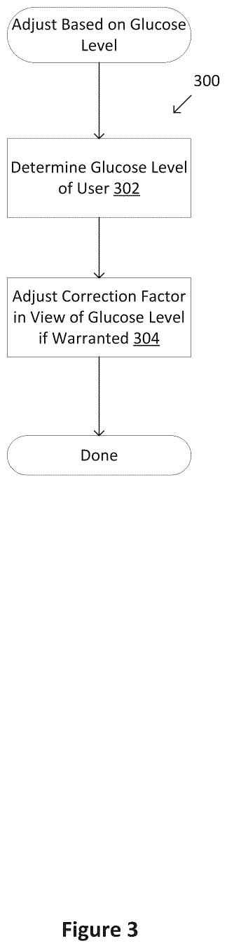

FIG. 3 depicts a flowchart 300 of illustrative steps that may be performed in exemplary embodiments to adjust the CF of the user based on glucose level of the user. At 302, the glucose level of the user may be determined. This may be the result of a reading by a sensor 106, like a glucose sensor such as a continuous glucose monitor (CGM). At 304, the CF of the user may be adjusted based upon the glucose level of the user if warranted. In some instances, no adjustment may be warranted, such as when the current glucose level of the user is not in a specified range that warrants adjustment, as detailed below.

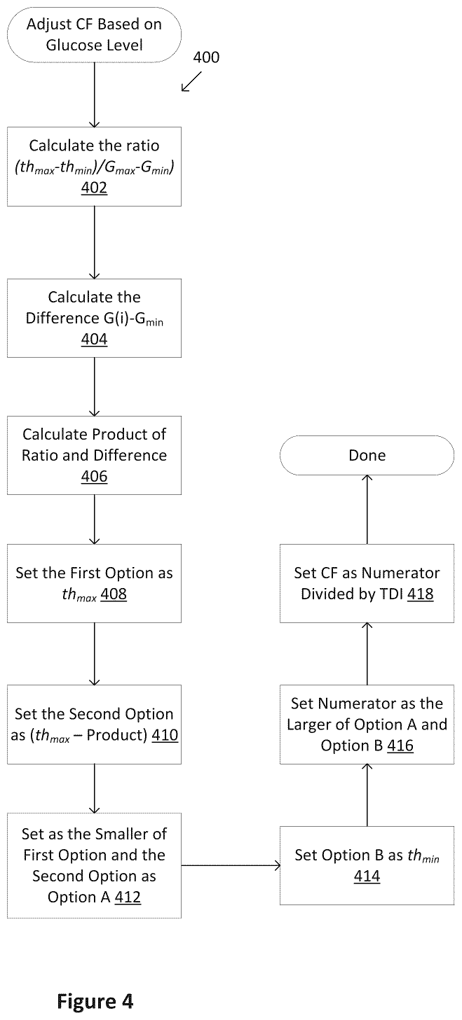

FIG. 4 depicts a flowchart 400 of illustrative steps that may be performed in exemplary embodiments in performing such an adjustment of the CF. In this approach, there may be a maximum glucose level threshold, designated as thₘₐₓ and a minimum glucose level threshold, designated as thₘᵢₙ. These thresholds represent maximum and minimum values that may be used as a numerator in an equation for determining the CF of the user, such as Equation 3 where the numerator 1800 is used. These threshold values specify the ends of a range over which the numerator may be varied to adjust the CF. In this example approach to adjusting the CF of the user, the CF may be scaled based on where the current glucose level of the user lies in that range. If the current glucose level is outside of the range, the CF of the user may be modified up to the value corresponding to the nearest boundaries of the range, in some embodiments.

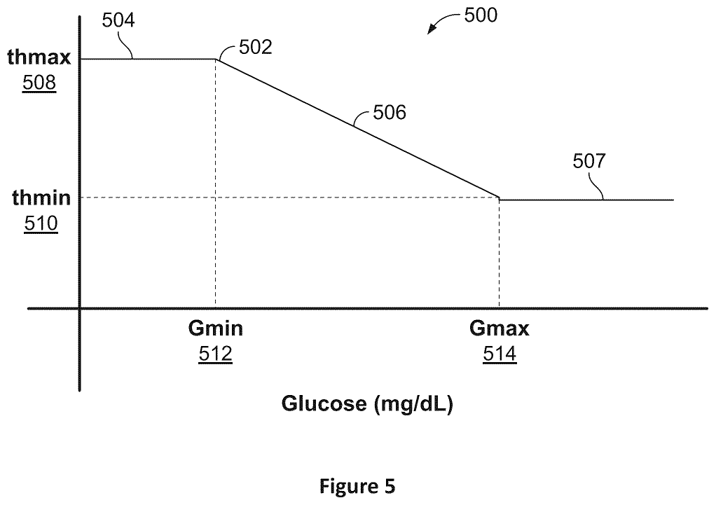

FIG. 5 depicts an illustrative plot 500 showing a linear scaling of the numerator over the glucose range. The plot 500 depicts a curve 502 of the numerator values. In exemplary embodiments, the numerator may not exceed thₘₐₓ 508 and may not be lower than thₘᵢₙ 510. In this exemplary embodiment, the scaling only occurs when the glucose level of the user is above Gₘᵢₙ 512. Hence, when the glucose level of the user is below Gₘᵢₙ 512, there is no scaling and as indicated by portion 504 of curve 502, and the numerator is set at thₘₐₓ 508 (e.g., 1800 mg/dL).

When the glucose level of the user is above Gₘₐₓ 514, the numerator may be set at thₘᵢₙ 510 (e.g., 1400 mg/dL) as indicated by portion 507 of the curve 502. For glucose level values of the user in between Gₘᵢₙ 512 and Gₘₐₓ 514, a linear scaling may be applied to the numerator as indicated by portion 506 of curve 502. In other embodiments, a quadratic scaling may be used, as explained in further detail below. An illustrative value for Gₘᵢₙ 512 is 180 mg/dL, and an illustrative value for Gₘₐₓ 514 is 300 mg/dL. That said, it should be appreciated that other values may be used for Gₘᵢₙ 512 and Gₘₐₓ 514 and for thₘᵢₙ 510 and thₘₐₓ 508. The practical effect of the scaling is to decrease the CF of the user for higher glucose levels, which, in turn, results in greater insulin dose calculations.

In one exemplary embodiment of scaling the CF value, a suitable equation for calculating a scaled value of the CF is:

$${CF}_{g} ( i ) = \qquad ( Equation 5 )$$

$$\frac{max ( min ( {th}_{max ,} {th}_{max} - ( \frac{{th}_{max} - {th}_{min}}{{G}_{max} - {G}_{min}} ) ( G ( i ) - {G}_{min} ) ) , {th}_{min} )}{TDI}$$

where CF_{g}(i) is the adjusted CF of the user based on glucose level of the user for cycle i.

With reference to FIG. 4, the adjustment to the CF of the user may be performed consistent with Equation 5 as follows. At 402, the ratio of (thₘₐₓ−thₘᵢₙ)/Gₘₐₓ−Gₘᵢₙ) may be calculated. This ratio specifies how much to adjust the numerator per unit of the range of glucose level values. At 404, the difference between the glucose level of the user at cycle I (i.e., G(i)) and the glucose minimum for the range where adjustments are made (i.e., Gₘᵢₙ) may be determined. This difference identifies where G(i) is in the range. At 406, the ratio calculated at 402 may be multiplied by the difference calculated at 404 to produce a product. At 408, thₘₐₓ may be set as a first option. At 410, the difference (thₘₐₓ−product calculated in 406) may be set as the second option. At 412, the smaller of the first option and the second option may be selected as option A. This operation at 412 ensures that the numerator does not exceed thₘₐₓ. At 414, thₘᵢₙ may be set as option B, and at 416, the larger of option A and option B may be selected as the numerator. The operation at 416 ensures that the numerator does not fall below thₘᵢₙ. At 418, the CF is calculated by dividing the numerator (representing the adjusted value in the range between 1800 and 1400, for example) by TDI of the user. Note that the value of this TDI term may be determined through a wide range of methods. This may simply be the sum of all insulin that was delivered in the previous 24 hours, or one day. Alternately, this may also be a value that is averaged across multiple days, i.e. average sum of total insulin delivered to the user each day over the past 2, 3, or more days. These calculations may be executed in a rolling basis, or may be calculated periodically and updated over fixed intervals. Further, the TDI may be calculated periodically by assessing different input values, such as the user's manually entered clinical parameters (basal) and converting them to equivalent daily insulin delivery requirements. Finally, the TDI may be directly entered by the user.

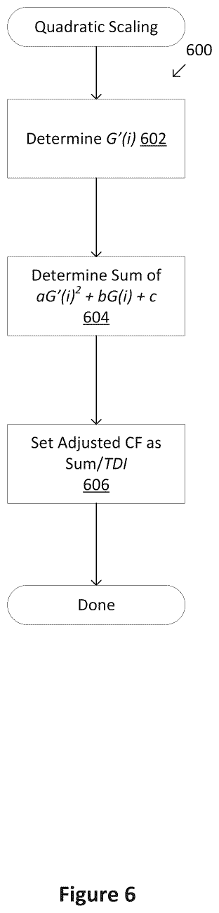

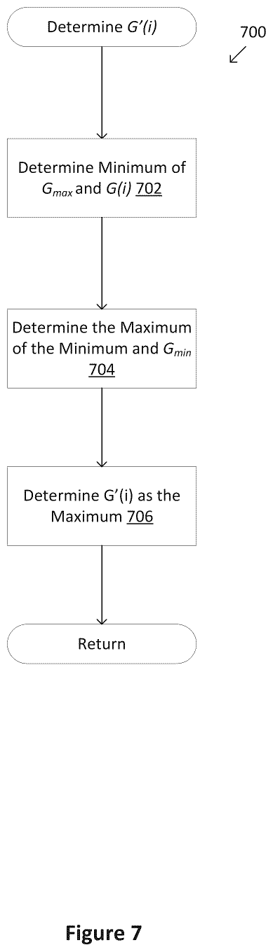

It should be appreciated that the scaling between thₘₐₓ and thₘᵢₙ, need not be linear. For example, the scaling may be quadratic. FIG. 6 depicts a flowchart 600 of illustrative steps that may be performed in exemplary embodiments to perform such quadratic scaling. At 602, G′(i) may be determined. G′(i) may be constrained to not assume a value greater than Gₘₐₓ and to not assume a value less than Gₘᵢₙ. FIG. 7 depicts a flowchart 700 of illustrative steps that may be performed in exemplary embodiments to determine G′(i). At 702, the minimum of Gₘₐₓ and G(i) may be determined. This operation prevents selection of a value greater than Gₘₐₓ. At 704, the maximum of the minimum and Gₘᵢₙ may be determined. At 706, G′(i) may be determined as the maximum. Thus, G′(i) is constrained to be between Gₘₐₓ and Gₘᵢₙ.

With reference to FIG. 6, at 604, a sum for the quadratic equation aG′(i)²+bG(i)+c may be determined, where a, b, and c are coefficients. One suitable form of this equation for quadratic scaling is:

$${CF}_{g , quad} ( i ) = \frac{0.03 {{G}^{′} ( i )}^{2} - 16.7 {G}^{′} ( i ) + 3900}{TDI} \qquad ( Equation 6 )$$

where CF_{g,quad}(i) is the quadratically scaled CF. At 606, the sum calculated in 606 is divided by TDI of the user to get the quadratically scaled CF.

In this example, the coefficients of the quadratic equation term may be fit through quadratic regression to match the example threshold and glucose maximum and minimum values that were given above. Different coefficients can also be calculated via quadratic regression for different targeted glucose value thresholds and limits of the heuristic rules of thumb.

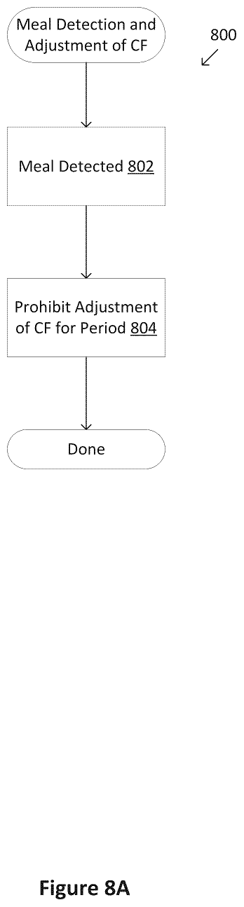

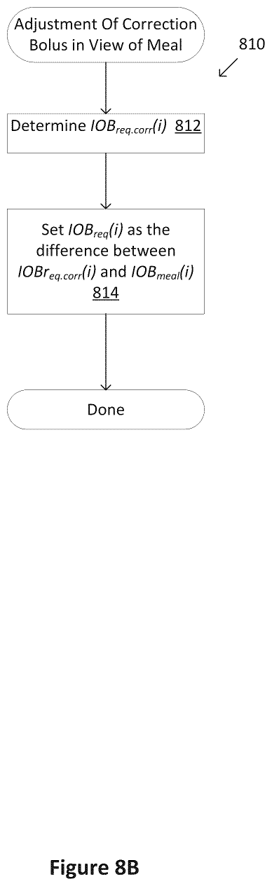

Such linear or quadratic scaling may not be desired after the user has recently consumed a meal since the user may have taken measures such as a meal bolus to compensate for the meal. As such, steps may be taken to account for meal ingestion by the user. FIG. 8A depicts a flowchart 800 of illustrative steps that may be performed to account for meal ingestion in exemplary embodiments. At 802, meal ingestion by the user may be detected. This may involve the user manually inputting information that the user is about to ingest a meal or recently has ingested a meal. This inputting of information may take the form of receiving an input or the user requesting a meal bolus of insulin or having recently delivered a meal bolus of insulin. Still further, the meal detection may be automatically detected, such as by observing the rate of change of the glucose level of the user of consecutive cycles or other consecutive time periods or by observing that a pattern of glucose level over consecutive periods matches a pattern indicative of meal ingestion. At 804, in response to the detection of the meal ingestion, the adjustment of the CF discussed above may be prohibited for a period, such as a 3- or 5-hour period or while blood glucose level is still elevated relative to a target glucose level.

In an alternate approach, instead of prohibiting adjustments to the CF of the user following meal ingestion, the adjusted CF may be used only for calculation of correction boluses. Specifically, the IOB required to bring the user to the target glucose level may be calculated as:

$${IOB}_{req} ( i ) = {IOB}_{req , corr} ( i ) = {IOB}_{meal} ( i ) \qquad ( Equation 7 A )$$

where IOBₘₑₐₗ(i) is the contribution to IOB of the meal bolus, IOB_{corr}(i) is the contribution to IOB of the correction bolus, and IOB_{regcorr}(i) is the IOB contribution of the correction bolus that is required to bring the glucose level of the user to the target glucose level, which may be calculated as

$${IOB}_{req , corr} ( i ) = \frac{G ( i ) - SP ( i )}{{CF}_{g}} - {IOB}_{corr} ( i ) \qquad ( Equation 7 B )$$

where CF_{g} is the CF of the user adjusted or scaled for current glucose level

FIG. 8B depicts a flowchart 810 of illustrative steps that may be performed in exemplary embodiments to adjust the correction bolus using the adjusted CF following a meal. At 812, IOB_{req,corr}(i) is determined, such as by using Equation 7B. At 814, the difference between IOB_{req,corr}(i) and IOBₘₑₐₗ(i) is calculated, and IOB_{req}(i) is set as the difference (see Equation 7A).

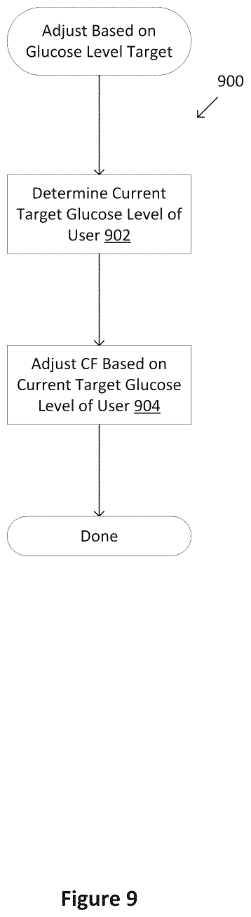

In some embodiments, the CF of the user may be adjusted based on the glucose level target of the user rather than the glucose level of the user. FIG. 9 depicts a flowchart 900 of illustrative steps that may be performed in exemplary embodiments to adjust the CF of the user based on the target glucose level of the user. At 902, the current glucose level target of the user may be determined. The user may set the current glucose level target in some exemplary embodiments. In other exemplary embodiments, the control application 116 or 120 may determine a suitable target glucose level of the user based on historic insulin delivery and/or glucose level data or may determine the target glucose level of the user based on one or more elements of information, such as age, gender, weight, etc. Still further, the control application 116 or 120 may, in some exemplary embodiments, adjust the target glucose level of the user over time based on glucose levels of the user, time in a desired range, etc. The CF of the user may be adjusted based on the current target glucose level of the user. Scaling may be performed for higher glucose level targets so that the control application 116 or 120 may be more aggressive in lowering the glucose level of the user. At 904, the CF is adjusted based on the current target glucose level of the user.

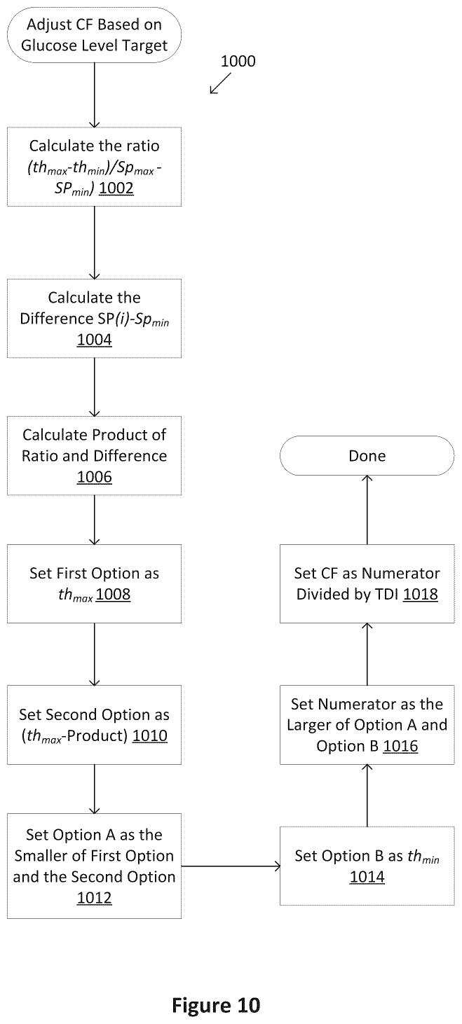

FIG. 10 depicts a flowchart 1000 of illustrative steps of one exemplary method that may be performed in exemplary embodiments to adjust the CF of the user based on the glucose level target of the user. These steps are consistent with using the following exemplary equation to adjust the CF of the user:

$${CF}_{SP} ( i ) = \qquad ( Equation 8 )$$

$$\frac{max ( min ( {th}_{max ,} {th}_{max} - ( \frac{{th}_{max} - {th}_{min}}{{SP}_{max} - {SP}_{min}} ) ( SP ( i ) - {SP}_{min} ) ) , {th}_{min} )}{TDI}$$

where CF_{SP}(i) is the adjusted CF of the user. This equation is similar to Equation 5 except that current target glucose level, target glucose level maximums and minimums replace glucose level, glucose level maximums and glucose level minimums. At 1002, the ratio (thₘₐₓ−thₘᵢₙ)/(SPₘₐₓ−SPₘᵢₙ) may be calculated. At 1004, the difference SP(i)−Spₘᵢₙ may be calculated. At 1006, the product of the ratio calculated in 1002 and the difference calculated in 1004 may be calculated.

At 1008, the first option is set as thₘₐₓ. At 1010, the second option may be set as (thₘₐₓ−product calculated at 1008). At 1012, option A may be set as the smaller of the first option and the second option. At 1014, option B may be set as thₘᵢₙ. At 1016, the numerator may be set as the larger of option A and option B. At 1018, the adjusted CF of the user may be set equal to the numerator divided by the TDI of the user.

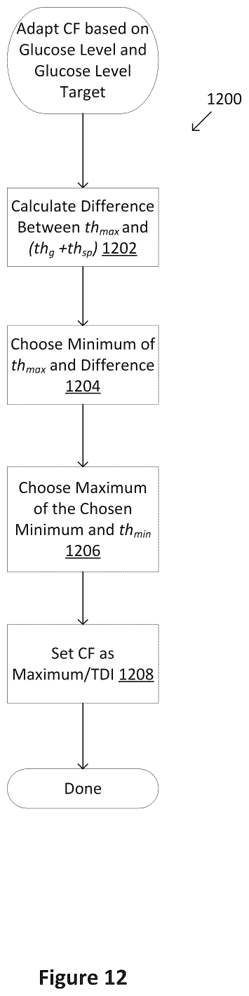

In some exemplary embodiments, the CF of the user may be adjusted based on both the glucose level of the user and the glucose level target of the user. FIG. 12 depicts a flowchart 1200 of illustrative steps that may be performed in exemplary embodiments to adjust the CF of the user based on both the current glucose level of the user and the current target glucose level of the user. A suitable equation for scaling the CF of the user based on both the current glucose level of the user and the current target glucose level of the user is:

$${CF}_{f} ( i ) = \frac{max ( min ( {th}_{max ,} {th}_{max} - {th}_{g} - {th}_{sp} ) , {th}_{min} )}{TDI} . \qquad ( Equation 9 )$$

At 1202, the difference thₘₐₓ−th_{g}−thₛₚ may be calculated. The threshold th_{g} may be calculated as:

$${th}_{g} = ( \frac{{th}_{max} - {th}_{min}}{{G}_{max} - {G}_{min}} ) ( G ( i ) - {G}_{min} ) . \qquad ( Equation 10 )$$

The threshold thₛₚ may be calculated as:

$${th}_{SP} = ( \frac{{th}_{max} - {th}_{min}}{{SP}_{max} - {SP}_{min}} ) ( SP ( i ) - {SP}_{min} ) . \qquad ( Equation 11 )$$

At 1204, the minimum between thₘₐₓ and the difference may be chosen. At 1206, the maximum of the chosen minimum of 1204 and thₘᵢₙ may be chosen. At 1208, the adjusted CF may be set equal to the maximum of 1206 divided by the TDI of user.

Another alternative is to adjust the CF of the user based on midpoints that is more robust against noise than the approach of Equation 9 and FIG. 12. For instance, the CF of the user may be adjusted based on the following:

$${CF}_{f} ( i ) = \frac{max ( min ( {th}_{max ,} \frac{( {th}_{max} - {th}_{G} ) + ( {th}_{max} - {th}_{sp} )}{2} ) , {th}_{min} )}{TDI}$$

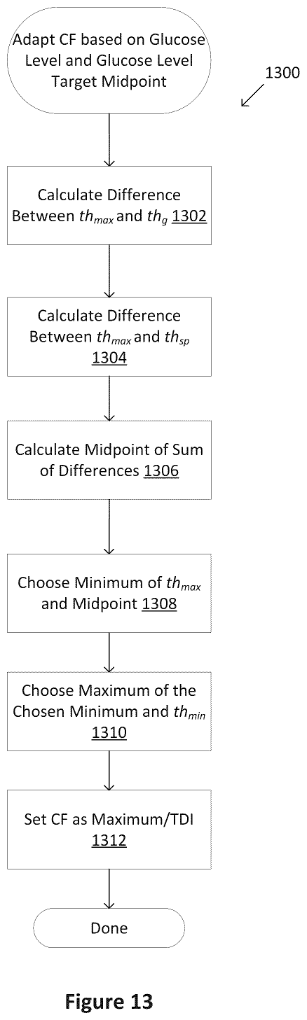

(Equation 12). FIG. 13 depicts a flowchart 1300 of the illustrative steps that may be performed in exemplary embodiments to determine the CF of the user per Equation 12. At 1302, the difference between thₘₐₓ and th_{G} may be determined. At 1304, the difference between thₘₐₓ and thₛₚ may be determined. At 1306, the differences calculated in 1302 and 1304 may be summed and divided by 2 to calculate the midpoint of the sum of the differences. At 1308, the minimum of thₘₐₓ and the midpoint may be chosen. At 1310, the maximum of the minimum chosen at 1308 and thₘᵢₙ may be chosen. At 1312, the maximum selected in 1310 may be divided by TDI and the resultant value may be set as the adjusted CF.

A further alternate approach that is more robust to noise than Equation 9 is:

$${CF}_{f} ( i ) = \frac{max ( min ( {th}_{max ,} {th}_{max} - {th}_{mid} ) , {th}_{min} )}{TDI} . \qquad ( Equation 13 )$$

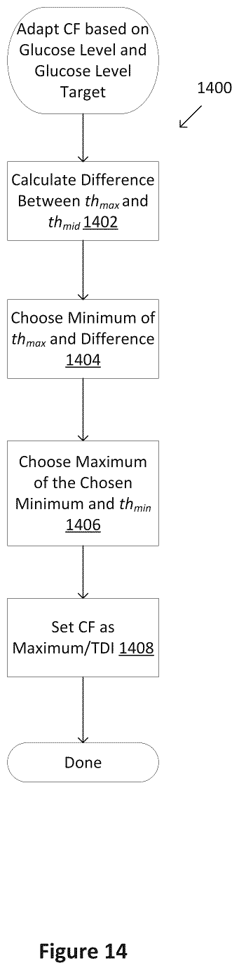

FIG. 14 depicts a flowchart 1400 of illustrative steps that may be performed in exemplary embodiments to determine an adjusted CF of the user based on Equation 13. At 1402, the difference between thₘₐₓ and th_{mid} may be determined. At 1404, the minimum between thₘₐₓ and the difference that was determined in 1402 may be chosen. At 1406, the maximum of the minimum chosen in 1404 and thₘᵢₙ may be chosen. At 1408, the adjusted CF may be set as the maximum that was chosen in 1406 divided by TDI.

The value th_{mid} that is referenced in Equation 13 may be calculated as:

$${th}_{mid} = ( \frac{{th}_{max} - {th}_{min}}{\frac{{G}_{max} - SP ( i )}{2} - \frac{{G}_{min} - SP ( i )}{2}} ) ( \frac{G ( i ) - SP ( i )}{2} ) . \qquad ( Equation 14 )$$

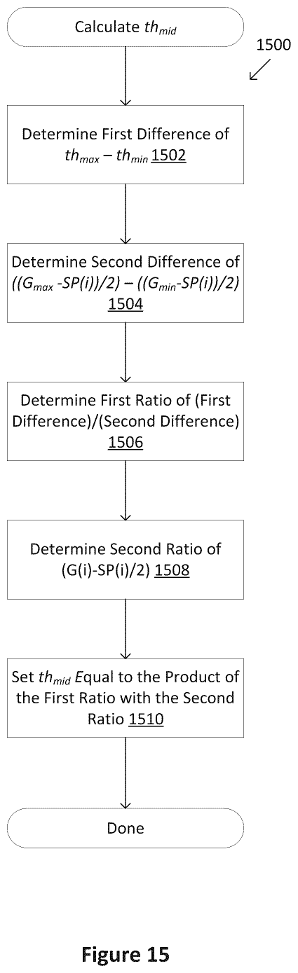

The value th_{mid} is the midpoint of the deviation between the current glucose level of the user and the current glucose level target of the user. FIG. 15 depicts a flowchart 1500 of illustrative steps that may be performed in exemplary embodiments to calculate th_{mid} using Equation 14. At 1502, a first difference between thₘₐₓ and thₘᵢₙ may be determined. At 1504, a second difference between (Gₘₐₓ−SP(i))/2 and (Gₘᵢₙ−SP(i))/2 may be determined. At 1506, a first ratio may be set equal to the first difference divided by the second difference. At 1508, a second ratio may be set equal to (G(i)−SP(i))/2. At 1510, th_{mid} may be set equal to the product of the first ratio with the second ratio.

While exemplary embodiments have been described herein, it should be appreciated that various changes in form and detail relative to the exemplary embodiments may be made without departing from the intended scope of the appended claims or equivalents thereof.

While exemplary embodiments have been described herein, it should be appreciated that various changes in form and detail relative to the exemplary embodiments may be made without departing from the intended scope of the appended claims or equivalents thereof.

1. A medicament delivery system for delivering medicament to a user, comprising:
a non-transitory storage medium storing computer programming instructions;
a processor configured for executing the computer programming instructions to cause the processor to:
determine a current glucose level of the user;
based on the determined current glucose level of the user, modify a current correction factor of the user; and
determine a dose of medicament to deliver to the user using the modified correction factor.

2. The medicament delivery system of claim 1, wherein the medicament delivery system comprises a medicament delivery device, and wherein the processor is part of the medicament delivery device.

3. The medicament delivery system of claim 1, wherein the computer programming instructions, when executed by the processor, cause the processor to define a range of glucose level values for which the current correction factor of the user will be modified and to determine whether the current glucose level of the user falls within the range.

4. The medicament delivery system of claim 3, wherein the modified correction factor is determined based upon where the current glucose level of the user falls within the range.

5. The medicament delivery device of claim 1, where the modified correction factor is set at a value equal to a numerator divided by total daily insulin of the user.

6. The medicament delivery system of claim 5, wherein the numerator is adjusted from a standard value to a modified value to modify the current correction factor of the user.

7. The medicament delivery system of claim 6, wherein the numerator may only be adjusted to a modified value that is in a range extending from the standard value to a minimum permitted value.

8. The medicament delivery system of claim 7, wherein the modified correction value is calculated using a scaled value relative to the standard value based upon where the current glucose level value of the user falls within the range.

9. The medicament delivery system of claim 8, wherein the modified scaled value is scaled linearly or quadratically relative to the standard value.

10. A medicament delivery system for delivering medicament to a user, comprising:
a non-transitory storage medium storing computer programming instructions;
a processor configured for executing the computer programming instructions to cause the processor to:
determine a current target glucose level of the user;
based on the current target glucose level of the user, modify a current correction factor of the user; and
determine a dose of medicament to deliver to the user using the modified correction factor.

11. The medicament delivery system of claim 10, wherein the computer programming instructions when executed by the processor cause the processor to define a range of target glucose levels for which the current correction factor of the user will be modified and to determine whether the current target glucose level of the user falls within the range.

12. The medicament delivery device of claim 10, where the modified correction factor is set at a value equal to a numerator divided by total daily insulin of the user.

13. The medicament delivery system of claim 12, wherein the numerator is adjusted from a standard value to a modified value to modify the current correction factor of the user.

14. The medicament delivery system of claim 13, wherein the numerator may only be adjusted to a modified value that is in a range extending from the standard value to a minimum permitted value.

15. The medicament delivery system of claim 14, wherein the modified correction value is calculated using a scaled value relative to the standard value based upon where the current glucose level value of the user falls within the range.

16. The medicament delivery system of claim 14, wherein the modified scaled value is scaled linearly or quadratically relative to the standard value.

17. A medicament delivery system for delivering medicament to a user, comprising:
a non-transitory storage medium storing computer programming instructions;
a processor configured for executing the computer programming instructions to cause the processor to:
determine a current glucose level of the user;
determine a current target glucose level of the user;
based on the current glucose level of the user and the current target glucose level of the user, modify a current correction factor of the user; and
determine a dose of medicament to deliver to the user using the modified correction factor.

18. The medicament delivery system of claim 17, wherein the modified correction factor is a scaled version of the current correction factor wherein the scaling is based on both where the current glucose level of the user falls within a range of glucose level values and where the current target glucose level falls with a range of target glucose level values.

19. The medicament delivery system of claim 17, wherein the medicament comprises insulin.

20. The medicament delivery system of claim 17, the correction factor is only modified if the current glucose level of the user exceeds the current glucose level target of the user.
